(19) United States
(12) Patent Application Publication
(10) Pub. No.: US 2009/0142370 A1
(43) Pub. Date: Jun. 4, 2009

(54) Method for preventing skin-cellular aging by using green algae extract and cosmetic composition containing green algae extract

(76) Inventors: Meng-Han Shih, Taipei City (TW); Mei-Fen Shih, Taipei City (TW)

Correspondence Address: CHARLES E. BAXLEY, ESQUIRE, 90 JOHN STREET, SUITE 309, NEW YORK, NY, 10038, US

(21) Appl. No.: 11/998,573

(22) Filed: Nov. 30, 2007

(51) Int. Cl. A61Q 19/08; A61P 17/00

(52) U.S. Cl. 424/195.17

(57) ABSTRACT

A method for preventing skin-cellular aging by using a green algae extract and a cosmetic composition containing the green algae extract are disclosed. An effective dose of the green algae extract is used to inhibit the activity of Metalloproteinase (MMP) in the fibroblast and to promote of collagens and elastins production in the fibroblast. The green algae extract can be mixed with a skin permeable cosmetic composition so that the cosmetic composition performs the effect of preventing extrinsic skin aging.

### BACKGROUND OF THE INVENTION

1. Technical Field

The present invention relates to a method and a composition for preventing extrinsic skin-cellular aging, which stems from excessive ultraviolet (UV) exposure. The present invention implements a green algae extract to prevent UV-induced skin problems such as cuticle thickening, rough texture, coarse wrinkles and flaccidness.

2. Description of Related Art

It was introduced in 2000 by Fisher et al. that UV irradiation damages human skin and causes premature skin aging (photoaging) characterized by cuticle thickening, rough texture, coarse wrinkles, and mottled pigmentation.

Also, Jenkins addressed in 2002 that repeated exposure to Ultraviolet-A (UVA) and Ultraviolet-B (UVB) radiation causes damage to human skin.

In 1999, Debelle et al. addressed that elastin and collagen are two major component of Extracellular matrix (ECM). The amount of elastin represents the youth appearance of skin. Collagen is responsible for the skin tone.

Fisher et al. later addressed the finding in 2002 that the expressions of various UV-induced metalloproteinases (MMPs) in skin fibroblasts lead to the breakdown of collagens and elastins and are thus related to photoaging in human skin.

Further, Fisherl and Watanabe et al. brought out in 1996 and 2004, respectively, that both UVA and UVB up-regulate MMP-1 in dermal fibroblasts via a PKC pathway.

Through the foregoing theories, it is learned that when UV makes inroads on skin, incomplete oxidation happens in fibroblasts and produces free radicals. MMP are activated by such free radicals. Unduly activated MMP leads to the breakdown of collagens and other ECM proteins, cell membranes, cell nucleuses in skin fibroblasts. Consequently, skin problems such as atrophic extracellular matrix, reduced thickness of corium, cuticle thickening, rough texture, coarse wrinkles and flaccidness are brought.

Conventional skin permeable formulations for external use employed to improve skin aging may contain the ingredients of alpha hydroxy acids (AHAs), Vitamin A Acid, Vitamin A or L-ascorbic acid. Alpha hydroxy acids help to remove aged keratinocytes and makes superficial improvement to cuticle thickening. Different definitions and regulations are applied to diverse concentration ranges of AHAs. For instance, the concentration of AHA products used for daily skincare may run below 20%, while those used by doctors for chemical peel can range from 20%-70%. Vitamin A Acid has the effects and functions similar to AHAs but is more irritant to skin. Improper use of Vitamin A Acid may cause side effects such as undue peel and inflamed skin. Thus, Vitamin A Acid shall only be applied under doctors' advice and serious sun-care. L-ascorbic acid possesses anti-oxidation ability that protects skin-cells from UV-induced injury and neutralizes free radicals. Moreover, L-ascorbic acid contributes to production of collagens so as to improve skin wrinkles and flaccidness.

### SUMMARY OF THE INVENTION

It is the main objective of the present invention to provided a material that is scientifically proven effective in preventing skin-cells from extrinsic aging and a skin permeable external formulation containing the material.

The material preventing skin-cells from extrinsic aging is a green algae extract. The present invention provides a method that employs an effective dose of the green algae extract to inhibit the activity of MMP in fibroblasts so as to promote proliferation of elastins and collagens in fibroblasts. The green algae extract can be mixed with a skin permeable cosmetic composition so that the cosmetic composition possesses the function of preventing skin-cells from extrinsic aging.

The disclosed method for preventing extrinsic skin-cellular aging involves using the green algae extract to inhibit MMP in fibroblasts.

The disclosed method comprises using the green algae extract to inhibit MMP in fibroblasts so as to promote proliferation of elastins and collagens in fibroblasts.

A preferable application of the present invention is to integrate an effective dose of the green algae extract into a cosmetic composition for human skin so as to endow the composition with ability to prevent extrinsic skin-cellular aging.

It is known that when skin receives excessive UV irradiation, MMP is activated in fibroblasts to break elastins and collagens. Thus, the present invention on the strength of the inhibitory effect of the green algae extract on MMP activity, provides the cosmetic composition integrated with the green algae extract as an ideal agent for protecting skin from UV-induced aging symptoms, such as cuticle thickening, rough texture, coarse wrinkles and flaccidness.

The dose of the green algae extract used in the present invention is not definitely limited and may vary in different preparation methods.

The green algae extract and the skin permeable composition integrated with the green algae extract of the present invention may be provided in any form, such as aqueous liquid, gel, lotion, cream, paste, powder and oily liquid. A preferred range for the green algae extract is in the range of about 0.01 to about 50% by weight of the total of composition.

### BRIEF DESCRIPTION OF THE DRAWINGS

The invention as well as a preferred mode of use, further objectives and advantages thereof, will best be understood by reference to the following detailed description of an illustrative embodiment when read in conjunction with the accompanying drawings, wherein:

FIG. 1 is a histogram, showing the inhibitory effects of the green algae extract of the present invention on PMA-induced MMP production;

FIG. 2 shows the inhibitory effects of the green algae extract of the present invention on PMA-induced MMP content by presenting MMP expression in fibroblasts analyzed by the Western blotting method;

FIG. 3 shows the augmenting effects of the green algae extract of the present invention on PMA-inhibited elastin expression analyzed by the Western blotting method;

FIG. 4 shows the inhibitory effects of the green algae extract of the present invention on MMP analyzed by RT-PCR method;

FIG. 5 is a histogram, showing MMP expression in fibroblasts after a total of 1.5 hours of UV exposure and the inhibitory effects of an effective dose of the green algae extract of the present invention on MMP activity;

FIG. 6 shows MMP and elastin expressions in fibroblasts after 30 minutes of UV exposure and the inhibitory effects on MMP activity as well as augmenting effects on elastins of the green algae extract of the present invention analyzed by the Western blotting method;

FIG. 7 shows that the green algae extract of the present invention inhibits expression of MMP gene in fibroblasts under UV exposure and promotes genetic expression of procollagens according to RT-PCR method;

FIG. 8 is an IR spectrum of the green algae extract of the present invention;

FIG. 9 is another IR spectrum of the green algae extract of the present invention; and

FIG. 10 is still another IR spectrum of the green algae extract of the present invention.

### DETAILED DESCRIPTION OF THE PREFERRED EMBODIMENT

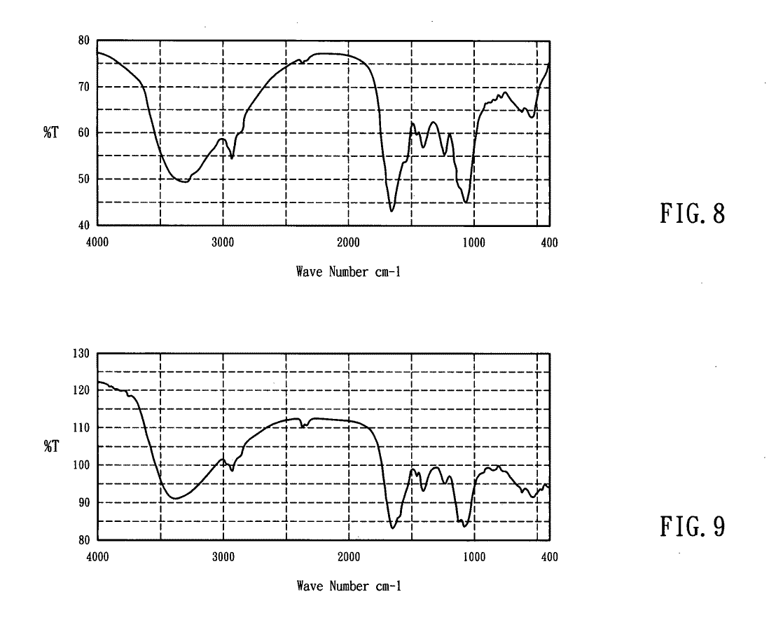

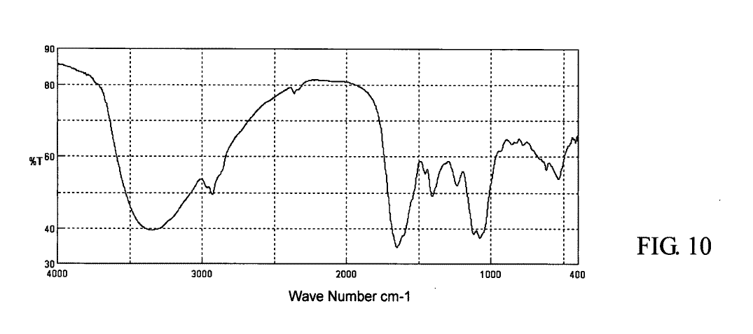

The present invention discloses a method for protecting skin-cells from extrinsic aging. The disclosed method provides a green algae extract as a major ingredient to inhibit MMP in fibroblasts and promote augmentation of elastins and procollagens mRNA. The green algae extract may be made through any commercially available method and mainly achieved by extracting aqueous liquid extract from water grown unicellular algae. From the IR spectrums of FIGS. 8 to 10, wherein the wave numbers are ranging from 4000 cm-1˜400 cm-1, it is learned that the green algae extract is substantially an organic compound and the major elements therein are carbon, hydrogen, as well as nitrogen. The concentrations of the elements are: nitrogen (N) 7.06%˜7.66%, hydrogen (H) 5.9%˜6.51%, and carbon (C) 35.67%˜38.1%.

For demonstrating the effects of the green algae extract on extrinsic skin cellular aging, the results of some experiments are provided for further illustration. The experiments are conducted under two kinds of conditions, wherein one uses phorbol 12-myristate 13-acetate (PMA) to induce skin-cells to produce MMP while the other uses UVB to induce skin-cells to produce MMP and the inhibitory effects of the green algae extract of the present invention on MMP produced under both conditions are tested.

[Using PMA to induce MMP in Fibroblasts]

### [Experiment 1]

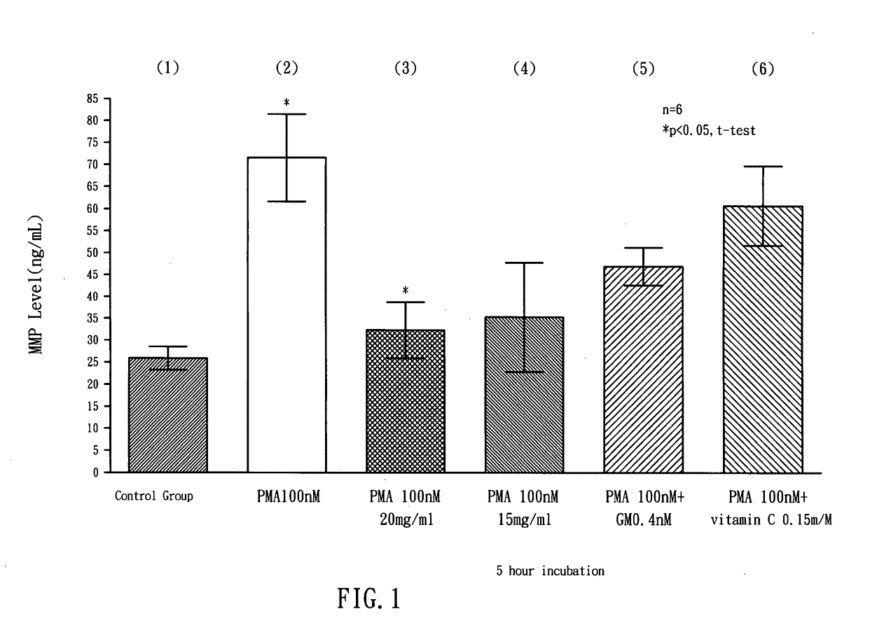

FIG. 1 shows the results of an experiment that demonstrates the inhibitory effects of the green algae extract of the present invention on PMA-induced MMP that quantified in the histogram. As known by those skilled in the art, PMA is an activator of protein kinase C (PKC) in fibroblasts, and PKC is an activator of MMP. Further, unduly activated MMP can break collagens as well as elastins in fibroblasts and in turn lead to aged skin. In the experiment shown in FIG. 1, Rectangle (1) belongs to a control group; Rectangle (2) belongs to a comparison group; and Rectangles (3), (4), (5) and (6) belong to an experimental group. A 5-hour incubation is conducted in each of the groups.

Rectangle (1), the control group, exhibits that after 5 hours of experimental period, fibroblasts release about 25 ng/mL of MMP.

Rectangle (2), the comparison group, exhibits that with 100 nM of PMA added, the average content of MMP rises up to about 70 ng/mL.

Rectangle (3) belongs to the experimental group and exhibits that with 100 nM of PMA and 20 mg/ml of the green algae extract added, the average content of MMP lowers to about 30 ng/mL.

Rectangle (4) belongs to the experimental group and exhibits that with 100 nM of PMA and 15 mg/ml of the green algae extract added, the average content of MMP lowers to about 35 ng/mL.

Rectangle (5) belongs to the experimental group and exhibits that with 100 nM of PMA and 0.4 nM of GM, a known non-selective MMP inhibitor, added, the average content of MMP is about 50 ng/mL.

Rectangle (6) belongs to the experimental group and exhibits that with 100 nM of PMA and 0.15 nM of the Vitamin C added, the average content of MMP is about 60 ng/mL.

The results of Experiment 1 demonstrates that the green algae extract is effective in inhibiting MMP activity and the higher effective dose achieves the better inhibitory effect. Through the data of Rectangles (3) and (4), it is evident that the inhibitory effects of the green algae extract of the present invention are substantially superior to those shown in other experimental groups.

### [Experiment 2]

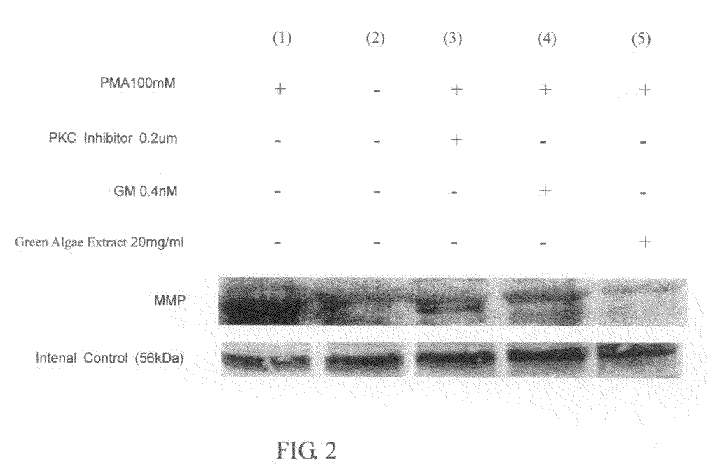

FIG. 2 shows the results of an experiment that demonstrates the inhibitory effects of the green algae extract of the present invention on PMA-induced MMP that analyzed by the Western blotting method. The images in the upper picture represent MMP expressions while the images in the lower picture are provided as a house keeping gene expressed protein.

Column (1) shows the result of using 100 nM of PMA in fibroblasts to induce production of MMP and demonstrates that MMP activity is increased.

Column (2) shows normal MMP activity in fibroblasts and may be taken as basal condition of the fibroblasts.

Column (3) shows the result of adding 100 nM of PMA and 0.2 um of PKC inhibitor in fibroblasts. As compared with the data of Column (1), PKC inhibitor slightly inhibits MMP activity.

Column (4) shows the result of adding 100 nM of PMA and 0.4 nM of GM the known MMP inhibitor, in fibroblasts. As compared with the data of Column (1), GM slightly inhibits MMP activity.

Column (5) shows the result of adding 100 nM of PMA and 20 mg/ml of the green algae extract in fibroblasts. As compared with the data of Columns (1), (2), (3) and (4), the green algae extract of the present invention significantly inhibits MMP activity.

Experiment 2 thus proves that the green algae extract of the present invention performs an outstanding effect on inhibiting MMP activity superior to the PKC inhibitor and GM, a known MMP inhibitor.

### [Experiment 3]

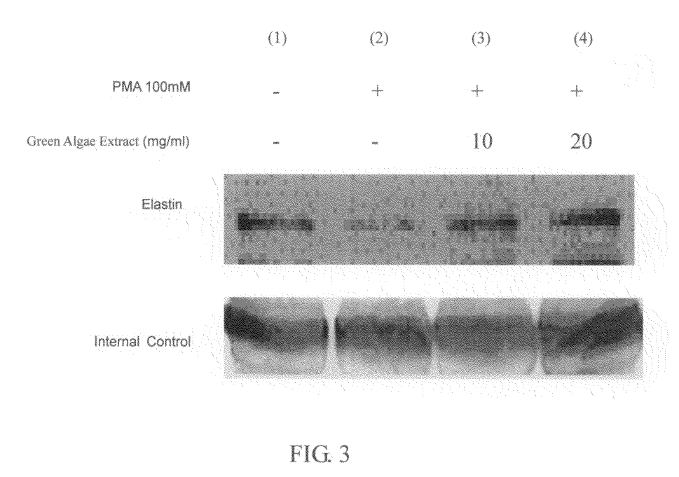

FIG. 3 shows the results of an experiment that demonstrates the ameliorating effects of the green algae extract of the present invention on elastins under the same condition as that MMP is activated by PMA analyzed by the Western blotting method. The images in the upper picture represent elastin protein while the images in the lower picture are provided as a house keeping gene expressed protein.

Column (1) shows normal elastin content in fibroblasts and may be taken as basal condition of the fibroblasts.

Column (2) shows that adding 100 nM of PMA in fibroblasts causes reduced elastins. This is because PMA activates MMP and unduly activated MMP breaks collagens and elastins in fibroblasts.

Column (3) shows the result of adding 100 nM of PMA and 10 mg/ml of the green algae extract of the present invention fibroblasts. As compared with the data of Column (2), the amount of elastins is significantly increased.

Column (4) shows the result of adding 100 nM of PMA and 20 mg/ml of the green algae extract of the present invention in fibroblasts. As compared with the data of Columns (2) and (3), the amount of elastins is more significantly increased.

The results of Experiment 3 demonstrates that the green algae extract is effective in proliferating elastins and the higher effective dose achieves the better proliferous effect.

### [Experiment 4]

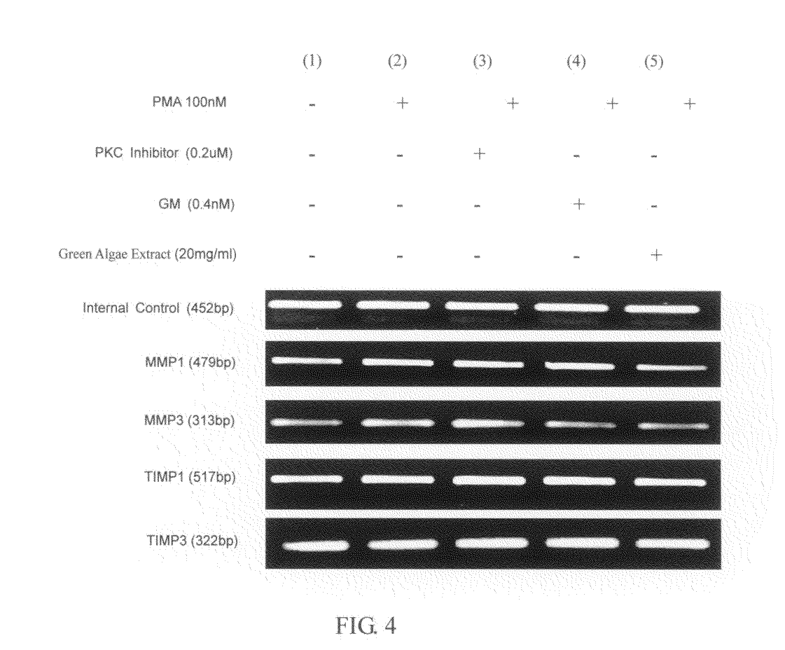

FIG. 4 shows the results of an experiment that demonstrates the inhibitory effects of the green algae extract of the present invention on the expression of MMP gene under the condition that MMP mRNA is activated by PMA analyzed by RT-PCR method. The intracellular inhibitor proteins TIMP1 and TIMP3 exist in fibroblasts to counterbalance metalloproteinases MMP1 and MMP3.

Column (1) shows normal metalloproteinases MMP1 and MMP3 and intracellular inhibitor proteins TIMP1 and TIMP3 in fibroblasts and may be taken as basal condition of the fibroblasts.

Column (2) shows the result of adding 100 nM of PMA in fibroblasts that leads to no so much change in intracellular inhibitor proteins TIMP1 and TIMP3 and enhanced genetic expressions of metalloproteinases MMP1 and MMP3.

Column (3) shows the result of adding 100 nM of PMA and 0.2 uM of PKC inhibitor in fibroblasts. As compared with the data of Column (2), PKC inhibitor fails to significantly enhance genetic expressions of intracellular inhibitor proteins TIMP1 and TIMP3 so that metalloproteinases MMP1 and MMP3 are not significantly reduced.

Column (4) shows the result of adding 100 nM of PMA and 0.4 nM of GM in fibroblasts. As compared with the data of Column (2), GM fails to significantly enhance genetic expressions of intracellular inhibitor proteins TIMP1 and TIMP3 so that metalloproteinases MMP1 mRNA, whereas MMP3 mRNA expression is less.

Column (5) shows the result of adding 100 nM of PMA and 20 mg/ml of the green algae extract of the present invention in fibroblasts. As compared with the data of Columns (1), (2), (3) and (4), the green algae extract of the present invention fails to significantly enhance genetic expressions of intracellular inhibitor proteins TIMP1 and TIMP3 but significantly reduces genetic expressions of metalloproteinases MMP1 and MMP3.

Experiment 4 thus proves that the green algae extract of the present invention performs better effect on inhibiting genetic expression of MMP than PKC inhibitor and GM.

[Using UV to induce MMP in Fibroblasts]

### [Experiment 1]

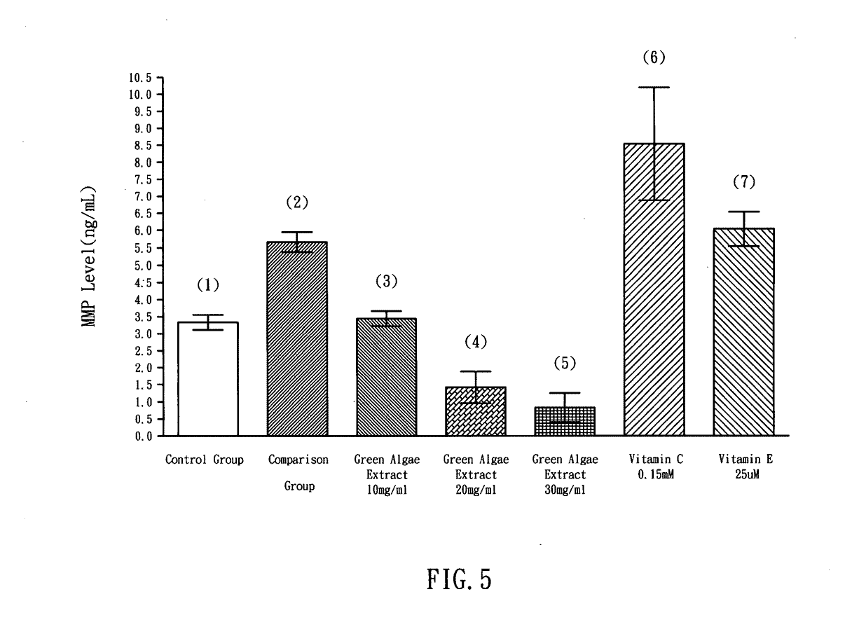

FIG. 5 shows the results of the experiment wherein MMP activity and inhibitory effects of an effective dose of the green algae extract of the present invention on MMP in fibroblasts after 1.5 hours of UVB exposure are tested and quantified in the histogram.

Rectangle (1) belongs to a control group and exhibits that fibroblasts release about 3.5 ng/mL of MMP without UV exposure.

Rectangle (2) belongs to a comparison group and exhibits that the average content of MMP in fibroblasts rises up to about 6.0 ng/mL after 1.5 hours of UV exposure.

Rectangle (3) belongs to an experimental group wherein the green algae extract of 10 mg/ml is added in fibroblasts before the fibroblasts are exposed in UV for 1.5 hours and exhibits that the average content of MMP is reduced to 3.5 ng/mL.

Rectangle (4) belongs to an experimental group wherein the green algae extract of 20 mg/ml is added in fibroblasts before the fibroblasts are exposed in UV for 1.5 hours and exhibits that the average content of MMP is reduced to 1.5 ng/mL.

Rectangle (5) belongs to an experimental group wherein the green algae extract of 30 mg/ml is added in fibroblasts before the fibroblasts are exposed in UV for 1.5 hours and exhibits that the average content of MMP is reduced to 1.0 ng/mL.

Rectangle (6) belongs to an experimental group wherein Vitamin C of 0.15 mM is added in fibroblasts before the fibroblasts are exposed in UV for 1.5 hours and exhibits that the average content of MMP is increased to 9.0 ng/mL.

Rectangle (7) belongs to an experimental group wherein Vitamin E of 25 uM is added in fibroblasts before the fibroblasts are exposed in UV for 1.5 hours and exhibits that the average content of MMP is maintained at 6.0 ng/mL.

The results of Experiment 1 demonstrates that the green algae extract is effective in inhibiting MMP activity and the higher effective dose achieves the better inhibitory effect. Through the data of Rectangles (3), (4) and (5), it is evident that the inhibitory effects of the green algae extract of the present invention are substantially superior to those shown in other experimental groups.

### [Experiment 2]

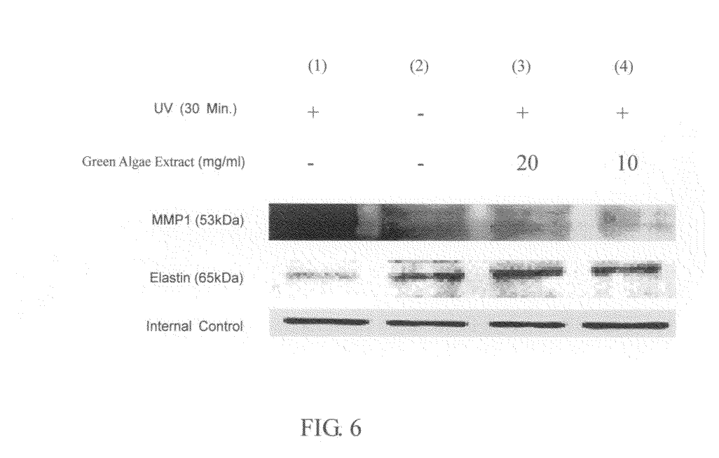

FIG. 6 shows the results of the experiment wherein the variation of MMP and elastins as well as augmenting effects of the green algae extract of the present invention on elastins in fibroblasts after 30 minutes of UV exposure are analyzed by the Western blotting method.

Column (1) belongs to a comparison group and exhibits that the average content of MMP significantly increases and the average content of elastins significantly decreased in fibroblasts after 30 minutes of UV exposure.

Column (2) belongs to a control group and exhibits contents of MMP and elastins in fibroblasts before UV exposure.

Rectangle (3) belongs to an experimental group wherein the green algae extract of the present invention of 20 mg/ml is added in fibroblasts before the fibroblasts are exposed in UV for 30 minutes. As compared with Column (1) of the comparison group, the present experimental group results a significantly decreased average content of MMP and a significantly increased average content of elastins in fibroblasts.

Rectangle (4) belongs to an experimental group wherein the green algae extract of the present invention of 10 mg/ml is added in fibroblasts before the fibroblasts are exposed in UV for 30 minutes. As compared with Column (1) of the comparison group, the present experimental group results a significantly decreased average content of MMP and a significantly increased average content of elastins in fibroblasts.

The results of Experiment 2 demonstrates that the green algae extract is effective in inhibiting MMP activity and enhancing elastins while the higher effective dose of the green algae extract achieves the better inhibitory effect on MMP activity and better augmenting effect on elastins.

### [Experiment 3]

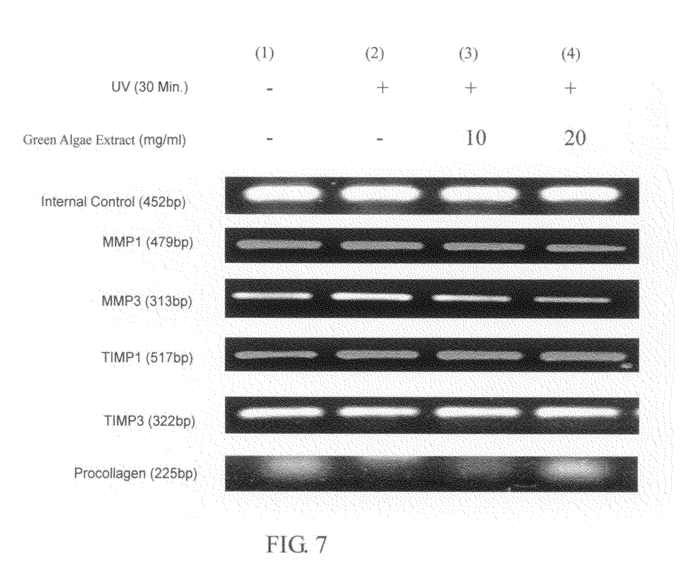

FIG. 7 shows the results of the experiment wherein the genetic expressions of MMP and Procollagens in fibroblasts after 30 minutes of UV exposure are analyzed by RT-PCR method. The intracellular inhibitor proteins TIMP1 and TIMP3 exist in fibroblasts to counterbalance metalloproteinases MMP1 and MMP3.

Column (1) belongs a control group and shows the genetic expressions of metalloproteinases MMP1 and MMP3, inhibitors TIMP1 and TIMP3 and Procollagens in fibroblasts without UV exposure.

Column (2) belongs to a comparison group wherein fibroblasts are exposed in UV for 30 minutes and exhibits that genetic expressions of metalloproteinases MMP1 and MMP3 are significantly increased while no significant variation occurs in genetic expressions of the inhibitors TIMP1 and TIMP3. However, genetic expressions of procollagens are decreased.

Column (3) belongs to an experimental group wherein the green algae extract of 10 mg/ml is added in fibroblasts before the fibroblasts are exposed in UV for 30 minutes. As compared with the data of the control group Column (1), and the comparison group Column (2), the green algae extract of the present invention reduces the genetic expressions of metalloproteinases MMP1 and MMP3, has no influence on the genetic expressions of inhibitors TIMP1 and TIMP3, but significantly increases the expressions of procollagens mRNA.

Column (4) belongs to an experimental group wherein the green algae extract of 20 mg/ml is added in fibroblasts before the fibroblasts are exposed in UV for 30 minutes. As compared with the data of the control group Column (1), and the comparison group Column (2), the green algae extract of the present invention reduces the genetic expressions of metalloproteinases MMP1 and MMP3, has no influence on the genetic expressions of inhibitors TIMP1 and TIMP3 and significantly increases the genetic expressions of procollagens.

The results of Experiment 3 demonstrates that the green algae extract is effective in inhibiting MMP genetic expression and increasing procollagens and the higher effective dose achieves the better inhibitory effect on the genetic expression of MMP and the better augmenting effect on procollagens.

The aforementioned experiments are for purposes of illustration and demonstration and not limitations to the practice of the present invention.

What is claimed is:

1. A method for preventing skin-cells from aging by using a green algae extract, comprising:
employing an effective dose of the green algae extract to inhibit an activity of an MMP in a fibroblast.

2. The method of claim 1, further comprising using an effective dose of the green algae extract to augmenting collagens and elastins in the fibroblast.

3. A cosmetic composition for protecting skin-cells from aging by using a green algae extract, endowed with an ability to inhibit an MMP activity in fibroblasts and containing an effective dose of the green algae extract to provide the ability to inhibit MMP.

4. The cosmetic composition of claim 3, wherein the green algae extract is an aqueous liquid extract extracted from fresh water grown unicellular algae.

5. The cosmetic composition of claim 3, wherein the cosmetic composition is in a form selected from the group consisting of aqueous liquid, gel, lotion, cream, paste, powder and oily liquid.

6. The cosmetic composition of claim 3, wherein the green algae extract is in a range of about 0.01 to about 50% by weight of the total cosmetics composition.

7. The cosmetic composition of claim 3, wherein the cosmetic composition is used to improve UV-induced fibroblast aging.

8. The cosmetic composition of claim 3, wherein the cosmetic composition is used to increase contents of collagens and elastins in fibroblasts.

9. A method for protecting skin-cells from aging, by using a green algae extract, comprising mixing an effective dose of the green algae extract in a cosmetic composition so as to endow an ability to inhibit MMP activity in fibroblasts to the cosmetic composition, to enhance a genetic expression of procollagens and to promote of elastin protein.
